(19) United States
(12) Patent Application Publication
(10) Pub. No.: US 2003/0059899 A1
(43) Pub. Date: Mar. 27, 2003

(54) Method for the hydrophobisation of DNA molecules

(76) Inventors: Murali Sastry, Maharashtra (IN); Ashavani Kumar, Maharashtra (IN); Vidya Ramakrishnan, Maharashtra (IN); Krishnarajanagar N. Ganesh, Maharashtra (IN)

Correspondence Address: NIXON & VANDERHYE P.C., 8th Floor, 1100 North Glebe Rd., Arlington, VA, 22201-4714, US

(21) Appl. No.: 09/962,602

(22) Filed: Sep. 26, 2001

(51) Int. Cl. C07H 21/04; C12P 19/34

(52) U.S. Cl. 435/91.2; 536/23.1

(57) ABSTRACT

The present invention relates to a method for the hydrophobisation of DNA molecules comprising mixing an aqueous solution of the DNA molecule with a solution of a cationic lipid or a surfactant in an organic solvent under agitation for a period in the range of 30 to 60 minutes to obtain the hydrophobic DNA in organic phase.

### FIELD OF THE INVENTION

The present invention relates to a method for the hydrophobisation of DNA molecules. More particularly, the present invention relates to a method for the extraction of DNA molecules of varying base sequences into a non-polar organic phase by electrostatic complexation with cationic fatty acids.

The cationic lipids present at the organic solvent-aqueous interface are also responsible for hybridization of single stranded oligonucleotides on extraction. The DNA molecules retain their double helical structure on phase transfer to non-polar organic phases indicating their possible applications in gene therapy

### BACKGROUND OF THE INVENTION

Hiydrophobised DNA is useful in various industrial applications. Upon hydrophobisation, the stability of the DNA is enhanced and does not require special conditions for storage below −20° C. Its stability can be verified by studying prominent features in emission bands in the visible region of the electromagnetic spectrum using UV and Fluorescence spectroscopy. DNA is useful for various applications such as identification of oligonucleotide sequences (gene sequencing) and in the treatment of genetic disorders by gene therapy.

Prior art processes for the hydrophobisation of DNA comprised forming DNA-cationic surfactant complexes in a Bligh and Dyer monophase [Reimer et al, (1995), Biochemistry, 34, 12877-12883]. In another prior art reference, it is disclosed that a solution containing equimolar amounts of DNA and cationic surfactant was water insoluble but was soluble in low-polar organic solvents [Tanaka, K., et al (1996), I. Am.Chem. Soc., 118, 10679; and Sergeyev, V. G., et al (1999), Langmuir, 15, 4434].

However, prior art methods of forming a DNA-cationic surfactant complex or a solution suffer from several disadvantages:

1. Pre-formed DNA molecules hydrophobised by Reimer et al above involves a three step process involving complexation of DNA with cationic surfactant in a Bligh and Dyer monophase and thereafter partitioning the monophase into a two phase system leading to transfer of DNA into the organic phase. This process requires a lot of maneuvering and is therefore complicated.

2. Previously studied protocols study only pre-formed DNA complexes for the hydrophobisation of DNA. In the process of the present invention, separate hybridization of oligonucleotide is not required since hydrophobisation is accompanied by hybridization.

3. The prior art reference of Tanaka and Sergeyev clearly suffer from a problem relating to solubility. The solutions containing DNA and cationic surfactant while insoluble in water are soluble in low polar organic solvents is therefore important to develop a process for the hydrophobisation of DNA molecules which overcomes the drawbacks enumerated above.

### OBJECTS OF THE INVENTION

It is an object of the invention to provide a simple and less complicated method for the hydrophobisation of DNA molecules.

It is another object of the invention to provide a method for the hydrophobisation of DNA molecules without the attendant problems of solubility.

It is a further object of the invention to provide a method for the hydrophobisation of DNA molecules which does not require a separate step of hybridization.

These and other objects of the invention is achieved by the method of the instant invention which involves a simple two phase shaking process for the complexing of DNA molecules with cationic lipid molecules.

### SUMMARY OF THE INVENTION

The present invention provides a simple two-phase shaking process by complexing DNA molecules with cationic lipid molecules at the hexane water interface and then phase transfer of the DNA into the organic phase. The invention is exemplified by hydrophobisation of naturally occurring DNA and synthetic DNA with different chain lengths, base sequences and different hybridizing properties for clinical applications and gene-transfer systems.

Accordingly, the present invention relates to a method for the hydrophobisation of DNA molecules comprising mixing an aqueous solution of the DNA molecule with a solution of a cationic lipid or a surfactant in an organic solvent under agitation for a period in the range of 30 to 60 minutes to obtain the hydrophobic DNA in organic phase.

In one embodiment of the invention, the DNA comprises synthetic DNA (10-60 mer), triple helical DNA, naturally occurring DNA such as calf thymus DNA or plasmid DNA.

In another embodiment of the invention, the lipid is selected from the group consisting of amine, ammonium salt or alcohol having C₈ to C₂₂ carbon atoms in the hydrophobic moiety.

In another embodiment of the invention the surfactant comprises a phospholipid having C₈ to C₂₂ carbon atoms in the hydrophobic moiety.

In a further embodiment of the invention, the organic solvent used comprises a polar organic solvent selected from the group consisting of hydrocarbons, substituted hydrocarbons, aromatic solvents, ethers, chloroform, aldehydes and ketones.

### DETAILED DESCRIPTION OF THE INVENTION

The present invention provides a simple two-phase shaking process by complexing DNA molecules with cationic lipid molecules at the hexane water interface and then phase transfer of the DNA into the organic phase. The invention is exemplified by hydrophobisation of naturally occurring DNA and synthetic DNA with different chain lengths, base sequences and different hybridizing properties for clinical applications and gene-transfer systems.

In the process of the invention, separate hybridization of oligonucleotide is not required since the hydorphobisation is accompanied by hybridization. Complementary single stranded DNA molecules in the bulk aqueous phase hybridise to form double helical structures during electrostatic complexation with cationic lipids at the liquid—liquid interface and are thereafter transferred to the organic phase under conditions where hybridization does not occur spontaneously in the bulk solution. In the instant invention, complexing of DNA molecules was done with not only low polar organic solvents but also with non-polar organic solvents such as hydrocarbons thereby accomplishing phase transfer in the non-polar organic phase.

Different organic solvents can be used. The solvents are generally selected from hydrocarbons, substituted hydrocarbons, aromatic solvents, ethers, chloroform, aldehydes and ketones.

The process of the invention is described below with reference to the examples which are illustrative and should not be construed as limiting the scope of the invention in any manner.

### EXAMPLE 1

This example illustrates the hydrophobisation of DNA (16 mer) with ODA molecules at the hexane-water interface and then phase transfer of DNA (16 mer) to the organic phase.

Oligonucleotides corresponding to the sequences GGAAAAAACTTCGTGC (ssDNA1), GCACGAAGTTTTTTCC (ssDNA2) and AGAAGAAGAAAAGAA (ssDNA3) were synthesized by β-cyanoethyl phosphoramidite chemistry on a Pharmacia GA plus DNA synthesizer, purified by FPLC and rechecked by RP HPLC ssDNA1 and ssDNA2 are complementary oligonucleotides while ssDNA3 is non-complementary to both ssDNA1 and ssDNA2. In typical experiments, 10 ml of a 10⁻⁴ M solution of ODA (Sigma Chemicals—used as received) in hexane was added to (a) 10 ml of 10⁻⁶ M aqueous solution of ssDNA1 and ssDNA2 taken in an equimolar ratio, (b) 10 ml of 10⁻⁶ M preformed double helical DNA molecules of ssDNA1 and ssDNA2 in water, and (c) 10 ml of 10−6 M aqueous solution of ssDNA1 and ssDNA3 taken in an equimolar ratio. The pH of the DNA solutions in all cases was 6.8. The hybridization of the complementary oligonucleotides ssDNA1 and ssDNA2 as well as the intactness of the double helical structure after phase transfer in the pre-formed duplex DNA experiment was followed using Fluorescence and UV-vis spectroscopic techniques.

### EXAMPLE 2

Calf Thymus (CT) DNA was hydrophobised with ODA and subsequently transferred to the organic phase. In this example, instead of synthetic DNA, naturally occuring calf thymus DNA was used. CT DNA was rendered hydrophobic by electrostatic complexation with ODA and transferred to the organic phase. The pH of the DNA solutions in all cases was 6.8. The intactness of the double helical structure after phase transfer in the calf thymus DNA experiment was followed using fluorescence and UV-vis spectroscopic techniques.

### EXAMPLE 3

10 ml of 10-4 M solution of tallow amine (Sigma Chemicals—used as received) in chloroform was added to 30 mer DNA in the following sequences: (a) 10 ml of 10⁻⁶ M aqueous solution of ssDNA1 and ssDNA2 taken in an equimolar ratio, (b) 10 ml of 10⁻⁶ M preformed double helical DNA molecules of ssDNA1 and ssDNA2 in water, and (c) 10 ml of 10⁻⁶ M aqueous solution of ssDNA1 and ssDNA3 taken in an equimolar ratio. Oligonucleotides corresponding to the sequences CCTTAAGCTTTTGTAQGAATCTATCTACATA (ssDNA1), GGAATTCGAAACATCTTAGTAGATGTAT (ssDNA2) AND AAGCGAATCGGGAGCAGCCTCGCACCGGGG (ssDNA3) were synthesized by β-cyanoethyl phosphoramidite chemistry on a Pharmacia GA plus DNA synthesizer, purified by FPLC and rechecked by RP HPLC. ssDNA1 and ssDNA2 are complementary oligonucleotides while ssDNA3 is non-complementary to both ssDNA1 and ssDNA2. The pH of the DNA solutions in all cases was 6.8. The hybridization of the complementary oligonucleotides ssDNA1 and ssDNA2 as well as the intactness of the double helical structure after phase transfer in the pre-formed duplex DNA experiment was followed using Fluorescence and UV-vis spectroscopic techniques.

### EXAMPLE 4

Hydrophobisation of plasmid DNA was done using ODA and then phase transferred to heptane (organic phase). The pH of the DNA solution in all the cases was 6.8.

### EXAMPLE 5

A 10 mer DNA was allowed to complex with lauryl amine (C₁₂) at the hexane-water interface making DNA hydrophobic. The hydrophobised DNA was then transferred to the organic phase. Oligonucleotides corresponding to the sequences GCATACATGT (ssDNA1), ACATGTATGC (ssDNA2) and GTGCACGCAT (ssDNA3) were synthesized by β-cyanoethyl phosphoramidite chemistry on a Pharmacia GA plus DNA synthesizer, purified by FPLC and rechecked by RP HPLC. ssDNA1 and ssDNA2 are complementary oligonucleotides while ssDNA3 is non-complementary to both ssDNA1 and ssDNA2. The pH of the DNA solutions in all cases was 6.8. The hybridization of the complementary oligonucleotides ssDNA1 and ssDNA2 as well as the intactness of the double helical structure after phase transfer in the pre-formed duplex DNA experiment was followed using Fluorescence and UV-vis spectroscopic techniques.

### EXAMPLE 6

This example illustrates the hydrophobisation of DNA (16 mer) with 1,2 dioleoyloxytrimethyl ammonium propane (DOTAP) at the hexane-water interface and then the phase transfer of DNA (16 mer) in the organic phase. DOTAP was sourced from Avanti Polar Lipids, Canada. Oligonucleotides of the sequences GGAAAAAACTTCGTGC (ssDNA1), GCACGAAGTTTTTACC (ssDNA2) and AGAAGAAGAAXAGAA (ssDNA3) were synthesized by β-cyanoethyl phosphoramidite chemistry on a Pharmacia GA plus DNA synthesizer, purified by FPLC and rechecked by RP HPLC. ssDNA1 and ssDNA2 are complementary oligonucleotides while ssDNA3 is non-complementary to both ssDNA1 and ssDNA2. In typical experiments, 10 ml of a 10⁻⁴ M solution of DOTAP in hexane was added to (a) 10 ml of 10⁻⁶ M aqueous solution of ssDNA1 and ssDNA2 taken in an equimolar ratio, (b) 10 ml of 10⁻⁶ M preformed double helical DNA molecules of ssDNA1 and ssDNA2 in water, and (c) 10 ml of 10⁻⁶ M aqueous solution of ssDNA1 and ssDNA3 taken in an equimolar ratio. The hybridization of the complementary oligonucleotides ssDNA1 and ssDNA2 as well as the intactness of the double helical structure after phase transfer in the pre-formed duplex DNA experiment was followed using Fluorescence and UV-vis spectroscopic techniques.

### ADVANTAGES OF THE INVENTION

1. The present invention provides a single step process making it faster, easier to handle and requiring less maneuvering. The need for separate hybridization of oligonucleotides is avoided since the hydrohobisation is accompanied by hybridization.

2. The process of the invention is a simple two phase shaking process without any attendant problems of solubility.

We claim:

1. A method for the hydrophobisation of DNA molecules comprising mixing an aqueous solution of the DNA molecule with a solution of a cationic lipid or a surfactant in an organic solvent under agitation for a period in the range of 30 to 60 minutes to obtain the hydrophobic DNA in organic phase.

2. A method as claimed in claim 1 wherein the DNA comprises synthetic DNA (10-60 mer), triple helical DNA, naturally occurring DNA such as calf thymus DNA or plasmid DNA.

3. A method as claimed in claim 1 wherein the lipid is selected from the group consisting of acne, ammonium salt or alcohol having C₈ to C₂₂ carbon atoms in the hydrophobic moiety.

4. A method as claimed in claim 1 wherein the surfactant comprises a phospholipid having C₈ to Ca₂₂ carbon atoms in the hydrophobic moiety.

5. A method as claimed in claim 1 wherein the organic solvent used comprises a polar organic solvent selected from the group consisting of hydrocarbons, substituted hydrocarbons, aromatic solvents, ethers, chloroform, aldehydes and ketones.
